(19) United States
(12) Patent Application Publication
(10) Pub. No.: US 2017/0258447 A1
(43) Pub. Date: Sep. 14, 2017

(54) ULTRASONIC TRANSDUCER AND OPERATION METHOD THEREFOR

(71) Applicant: ALPINION MEDICAL SYSTEMS CO., LTD., Hwaseong-si, Gyeonggi-do (KR)

(72) Inventors: Hyungkeun LEE, Daegu (KR); Keonho SON, Seongnam-Si (KR)

(73) Assignee: ALPINION MEDICAL SYSTEMS CO., LTD., Hwaseong-si, Gyeonggi-do (KR)

(21) Appl. No.: 15/329,852

(22) PCT Filed: Jul. 31, 2014
(86) PCT No.: PCT/KR2014/007078

§ 371 Date: Jan. 27, 2017

(51) Int. Cl. A61B 8/00

(52) U.S. Cl. CPC A61B 8/4483; A61B 8/4461

(57) ABSTRACT

The present disclosure provides an ultrasonic transducer including a swivel member, a linking member and an array. The swivel member rotates upon receiving an actuating force and has a thrust mechanism. The linking member is connected to the swivel member to make a movement along a pendulum motion path upon receiving thrust generated by the thrust mechanism of the swivel member. The array is connected to the linking member to rotate a predetermined angle upon receiving thrust generated by the movement of the linking member. In some embodiments, the swivel member has an insertion pin coupled to the linking member including a guide groove for receiving the insertion pin, and the guide groove length is greater than the insertion pin diameter. The array includes a stop for restraining the linking member from moving side to side. The present disclosure also provides a method of operating the aforementioned ultrasonic transducer.

### TECHNICAL FIELD

The present disclosure relates to an ultrasonic transducer having a simple structure and capable of a precise array rotation, and an operation method therefor.

### BACKGROUND

The statements in this section merely provide background information related to the present disclosure and do not necessarily constitute prior art.

Generally, the ultrasound diagnostic system is adapted to irradiate ultrasonic signal waves from the surface of a subject toward an internal target region of the subject and extract information from the reflected ultrasonic signal waves, to non-invasively obtain an image on the single layer or blood flow in soft tissue.

Such ultrasound diagnostic system, when compared with other imaging instrument, e.g., an X-ray inspection apparatus, CT scanner (Computerized Tomography Scanner), MRI scanner (Magnetic Resonance Image Scanner) and nuclear medical examination apparatus, is compact and inexpensive, and capable of real time display. Taking the high level safety advantage of involving no exposure to X-ray and other radiations, the ultrasound diagnostic system has been widely used for cardiac, abdominal, urinary and genital tract diagnoses.

In particular, the ultrasound diagnostic system comprises an ultrasonic transducer for transmitting an ultrasonic signal to a subject to obtain an ultrasound image of the subject and receiving ultrasound signals which have been reflected from the subject.

For obtaining an ultrasound image or treatment of the subject, the ultrasound transducer radiates ultrasonic waves to the treatment site and receives ultrasonic echo waves that have been reflected from the subject.

One of the methods for an ultrasonic transducer to generate ultrasonic waves is to use the properties of a piezoelectric material. A piezoelectric material is a substance for converting mechanical energy to electrical energy or vice versa. For example, a piezoelectric member for use in ultrasonic transducers is formed with a top electrode and a bottom electrode which are responsive to an application of power for vibrating the piezoelectric member which then causes the interconversion between the electrical signal and the acoustic signal.

Ultrasonic transducers generally include an image transducer that serves the purpose of image acquisition of a subject, a HIFU (high intensity focused ultrasound) transducer for the purpose of treatment of a subject, and a hybrid of a image transducer and a HIFU transducer for carrying out the diagnosis and treatment at the same time.

Ultrasonic transducers typically have a part referred to as an array that radiates ultrasonic waves. For the sake of expanding diagnostic or treatment capability of an ultrasonic transducer over a subject and for an easier use thereof, the ultrasonic transducer may be made to have its array configured to rotate.

Typical arrangements for rotating the array, as disclosed in Japanese Laid-Open Patent Publication No. 2006-006491 and Korea Patent Publication No. 2012-0088642, utilize power transmission mechanism including gears, wires, cams, belts, etc. in the ultrasonic transducer. However, the use of such power transmission mechanism requires a complex driving mechanism for the sake of a precision rotation of the array. This leads to an ultrasonic transducer structure that is bulkier and more complicated to manufacture.

### DISCLOSURE

### Technical Problem

Accordingly, the present disclosure in some embodiments seeks to provide an ultrasonic transducer having a simple structure and being capable of precise array rotation and an operation method therefor.

The present disclosure addresses other technical issues than and not limited to that mentioned above, and other unmentioned technical issues will be clearly understood by those of ordinary skill in the art to which the present disclosure pertains from the following description.

### SUMMARY

In accordance with some embodiments of the present disclosure, an apparatus comprises an ultrasonic transducer including a swivel member, a linking member and an array. The swivel member is configured to rotate upon receiving an actuating force and to have a thrust mechanism. The linking member is configured to be connected to the swivel member so as to make a movement along a pendulum motion path upon receiving a thrust generated by the thrust mechanism of the swivel member. The array is configured to be connected to the linking member so as to rotate a predetermined angle upon receiving a thrust generated by the movement of the linking member.

According to at least one embodiment of the disclosure, the driving unit includes a motor.

According to at least one embodiment of the disclosure, the thrust mechanism of the swivel member includes an insertion pin coupled to the linking member that includes a guide groove configured to receive the insertion pin, and the guide groove has a length which is greater than a diameter of the insertion pin. In addition, the array according to at least one embodiment of the present disclosure includes a stop for restraining the linking member from moving side to side.

According to the disclosed configuration of embodiments, the linking member is configured to receive a thrust generated by a swivel motion of the swivel member to generate a linear motion as viewed in plan view.

In addition, according to some embodiments of the disclosure, the linking member has a through hole, the array comprises a support shaft configured to be inserted the through hole, and the array further comprises a coupler coupled to its external housing.

According to the disclosed configuration of embodiments, the coupler has an axis about which the array is rotated under a thrust generated by the movement of the linking member.

In accordance with some embodiments of the present disclosure, an ultrasonic transducer includes a swivel arm, a link and an array. The swivel arm is configured to rotate upon receiving an actuating force and to include an insertion pin. The link is configured to include a guide groove for receiving the insertion pin of the swivel arm and to have a through hole. The array is configured to include a support shaft that is inserted in the through hole of the link. Herein, the guide groove has a length which is greater than a diameter of the insertion pin, and the array includes a stop for restraining the link from moving side to side, the ultrasonic transducer further comprises a housing configured to receive the array, the array further comprises a coupler coupled to the housing at sides of the housing, and thereby the insertion pin of the swivel arm rotates for causing the link forming the guide groove to exert a linear motion, and upon receiving a thrust generated by the linear motion of the linking member, the array is rotated by a predetermined angle about an axis of the coupler.

In addition, the present disclosure provides a method of operating an ultrasonic transducer, including rotating a swivel member with an actuating force from a driving unit, moving a linking member linearly as viewed in plan view along a pendulum motion path with a thrust generated by a rotation of the swivel member, through a thrust mechanism provided in the swivel member, and rotating an array connected to the linking member by a predetermined angle with a thrust generated by a linear motion of the linking member.

A method of operating an ultrasonic transducer method according to at least one embodiment of the present disclosure may be embodied by respective functions and steps performed by the configurations of the ultrasound transducer described above.

### ADVANTAGEOUS EFFECTS

The present disclosure has the following effects.

(1) The present disclosure provides novel and advanced configuration and operation of an ultrasonic transducer wherein a linking member is moved with a thrust generated by the rotation of a swivel member, and the movement of the linking member causes an array to swivel.

(2) The present disclosure advantageously needs to adopt a configuration or a component that simply performs a swivel motion and linear motion, which is implemented by a structure that is way simpler than the prior art and manufactured more easily, resulting in reduced production cost.

(3) The present disclosure can control the rotational angle of the array variously and accurately in a predetermined range, so as to visualize precise three-dimensional images.

(4) The present disclosure provides a driving unit serving as a driving source arranged coplanar with an array, and has components compacted in a casing and a housing, featuring ease of use, lighter weight product, and design simplicity.

It should be understood that the above-described effects of the disclosure are intended to be illustrative only and not limited thereto.

### [BRIEF DESCRIPTION OF THE DRAWINGS]

FIG. 1 is a perspective view showing an ultrasonic transducer according to at least one embodiment of the present disclosure.

FIG. 2 is an exploded view showing an ultrasonic transducer according to at least one embodiment of the present disclosure.

FIG. 3 is a bottom perspective view of a link and an array of the ultrasonic transducer of FIGS. 1 and 2, with a swivel arm removed for convenience.

FIG. 4 is a bottom perspective view of the swivel arm omitted in FIG. 3.

FIG. 5A is a vertical cross-sectional view of a swivel arm and a link in accordance with at least one embodiment of the present disclosure.

FIG. 5B is a horizontal cross-sectional view showing a coupled state of an insert pin and a guide groove of FIG. 5A.

FIG. 5C is a conceptual diagram showing operation in a plan view to explain the principles of the rotation of a swivel arm and the linear movement of a link, according to at least one embodiment of the present disclosure.

FIG. 6 is a perspective view showing a coupling structure of a link with an array, according to at least one embodiment of the present disclosure.

FIG. 7(A-C) is a conceptual side view illustrating an array swiveling in accordance with the linear movement of a link, in accordance with at least one embodiment of the present disclosure.

FIG. 8 is a perspective view of coordinate axes (A, C, Y) given to a transducer according to at least one embodiment of the present disclosure.

FIG. 9 is a conceptual diagram illustrating the relationship between the moving distance of a link and the rotational angle of a swivel arm, according to at least one embodiment of the present disclosure.

FIG. 10 is a conceptual diagram illustrating the relationship between the moving distance of a link and the rotational angle of an array, in accordance with at least one embodiment of the present disclosure.

FIG. 11 is a graph for explaining analysis results of the rotation angles of a swivel arm and an array, according to at least one embodiment of the present disclosure.

FIG. 12 is a flowchart showing the operating sequence of an ultrasonic transducer according to at least one embodiment of the present disclosure.

### DETAILED DESCRIPTION

With reference to the accompanying drawings, a description will be given of at least one embodiment according to the present disclosure.

The present disclosure encompasses various modifications and can take a variety of forms of which some specific embodiments will be illustrated in the drawings and detailed in the text. However, this is not intended to limit the present disclosure by the particular form disclosed, and it should be understood that the present disclosure covers all modifications, equivalents, and substitutes included in the idea and scope of the present disclosure. In the following description, similar reference numerals is designated to similar elements, although the elements are shown in different drawings. In the accompanying drawings, the dimensions of the structures might be shown exaggerated for clarity of the disclosure or abridged for a schematic repesentation of the configurations of some embodiments.

In addition, terms such as first and second may be used to describe various elements, although the above elements shall not be bound by the above terms. The above terms are used only to distinguish one element from the other. For example, the first element may be renamed as a second element without departing from the scope of the disclosure, and similarly a second element may be renamed as a first element. On the other hand, if not otherwise defined, all terms as used herein, including technical and scientific terms, have the same meaning as commonly understood by one of ordinary skill in the art.

Any term that is defined in a general dictionary shall be construed to have the same meaning in the context of the related art, and shall not be construed ideally or excessively formalistically unless the present disclosure expressly defines them so.

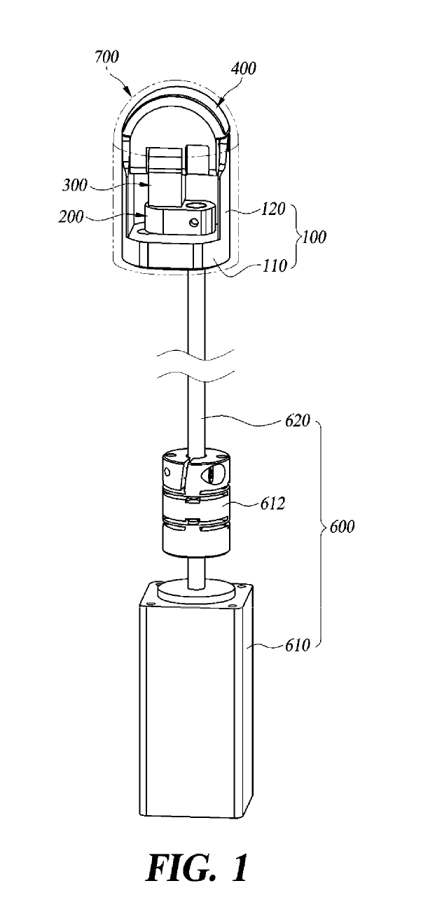

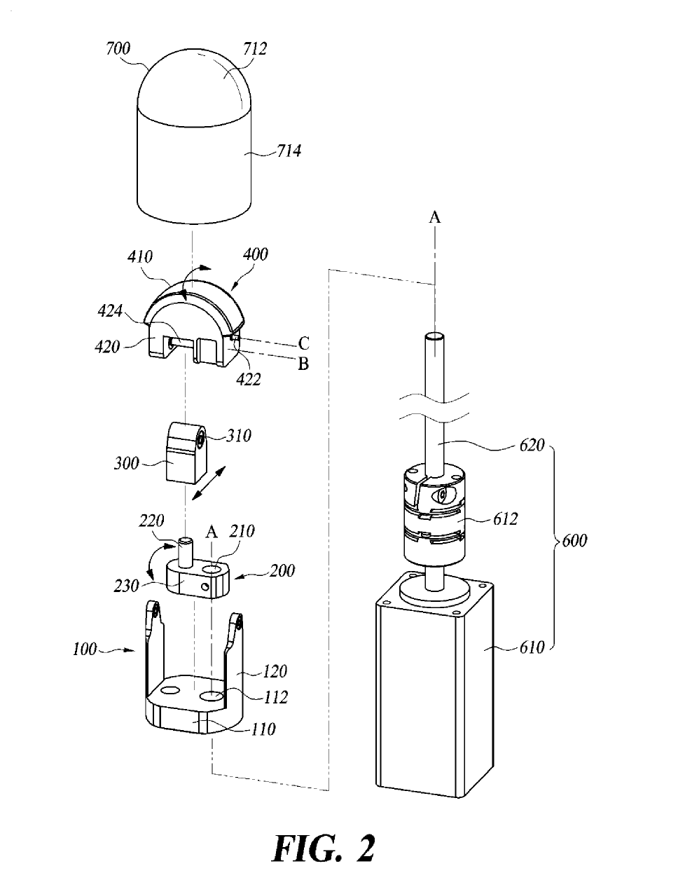

FIG. 1 is a perspective view showing an ultrasonic transducer according to at least one embodiment of the present disclosure. FIG. 2 is an exploded view showing an ultrasonic transducer according to at least one embodiment of the present disclosure.

First, the ultrasonic transducer of the present disclosure in FIG. 1 roughly includes a housing 100, a swivel arm 200, a link 300, an array 400 and a driving unit 600. As described in detail below, when the swivel arm 200 is rotated by the driving of the driving unit 600, the rotational force is converted into a thrust movement of the link 300, which rotates the array 400 by a predetermined angle.

The swivel arm 200 and the link 300 are termed to exemplify a swiveling member and a connecting member, respectively as defined in the claims of the present disclosure, but the terms are not intended to limit the scope of the disclosure.

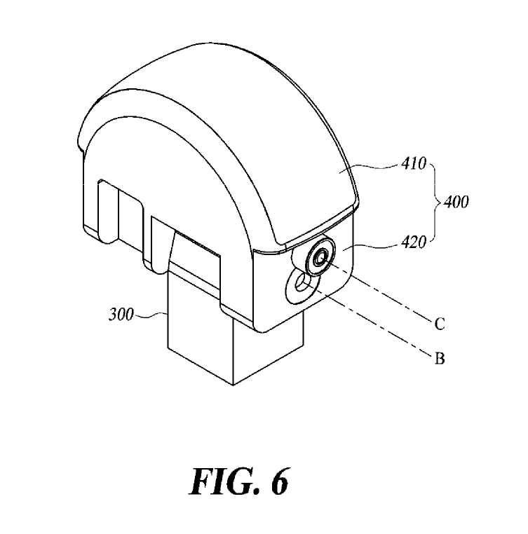

The driving unit 600 includes a motor housing 610 with a built-in motor and a shaft 620 that interconnects with an output shaft of the motor as shown in FIG. 6. Reference numeral 612 denotes a weight which is installed adjacent to the housing 610 so that the shaft 620 fully supports its upper members which are the housing 100, the swivel arm 200 and the link 300 and that the shaft 620 delivers a stable rotational force thereto. The weight and the number of installed weights 612 are preferably determined to achieve a balance with the total weight of the upper members above the driving unit 600. The shaft 620 of the driving unit 600 is elongated in the vertical direction.

The swivel arm 200, the link 300 and the array 400 of the ultrasonic transducer of some embodiments are housed compactly in a space formed by the housing 100 and a casing 700. The housing 100 is composed by a base 110 that constitutes the housing bottom and a pair of upright sides 120 formed on either side of the base 110 and integrally with the base 110. In FIG. 2, the base 110 according to some embodiments is formed with a through-hole 112 disposed at an off-centered location. The diameter of the through hole 112 is appropriate for the shaft 620 to pass through without interference. Alternatively, the shaft 620 and the through-hole 112 may be arranged to be fitted in close contact with smooth relative rotation maintained therebetween by using an interposed hub bearing or the like. The base 110 is substantially rectangular in shape with the corners notched as a mere example in some embodiments. Besides, the base 110 may be generally formed in a circular shape or others, and it may be randomly modified in terms of form, size and number.

The casing 700 in some embodiments has domed or arcuate upper portion 712 made of any material such as a transparent plastics material and the like as long as they can transmit ultrasound. The casing 700 has a cylindrical lower portion 714 which extends as far as to fit in full contact with the sides 120 of the housing 100 down to the bottom of the base 110.

With this configuration, the housing 100 and the casing 700 provide the space for housing the swivel arm 200, the link 300 and the array 400 in a compact, integrated package, as described above.

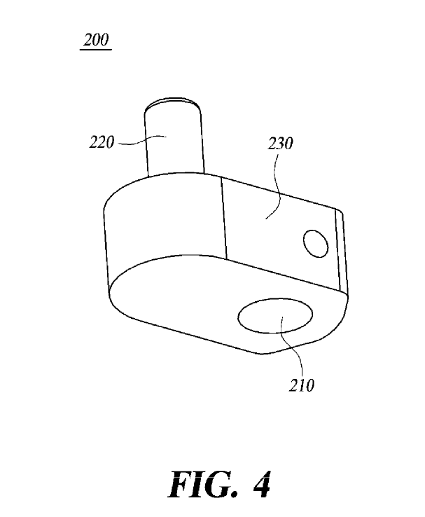

The swivel arm 200 of some embodiments will be described with reference to FIG. 2 and FIG. 4. The swivel arm 200 includes a body 230 extending laterally and terminating with arcuate right and left side walls, and a through-hole 210 formed in a position axially aligned with the abovementioned through-hole 112. A good diameter for the through hole 210 is substantially equal to or slightly larger than the diameter of the shaft 620 so that the distal end of the shaft 620 after passing through the through-hole 112 is tightly press fitted into the through hole 210. Alternatively, the shaft 620 is distally threaded and the through hole 210 is corespondingly threaded so that they can be configured to to be screwed together while the two sets of threads intermesh.

In addition, the body 230 has an insertion pin 220 extending upright in a position diametrically opposed to the through-hole 210. The insertion pin 220 functions as a thrust mechanism.

In FIG. 2, “A” is the center axis about which the swivel arm 200 rotates, and it comes into line with the longitudinal center line of the shaft 620. With the shaft 620 passing through the through hole 112 into an engagement with the through-hole 210, rotating the shaft 620 by the motor in the clockwise or counterclockwise direction turns the rotational arm 200 including the insertion pin 220 in the directions of arrows as shown in FIG. 2 about the shaft 620, that is, the axis A.

Good material for the housing 100 and the swivel arm 200 in some embodiments is a rigid material including aluminum, stainless steel and such metals or molded plastic. The swivel arm 200 in operation is prevented from rotational frictions or interferences by spacing the bottom of the body 230 at a slight distance from the upper surface of the base 112 or by periodically applying a lubricant such as grease to the body 230 and the base 112 where they contact each other.

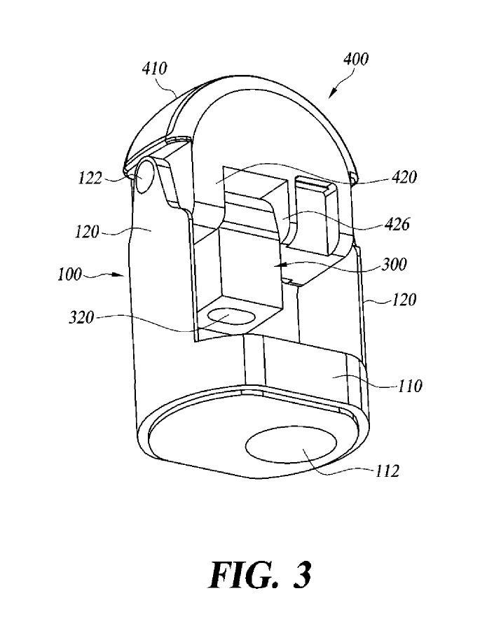

Now, referring to FIGS. 2 and 3, a link 300 is illustrated as operatively connected to the swivel arm 200 in some embodiments of the present disclosure. In FIG. 3, the swivel arm is omitted for convenience of illustration.

The link 300 has an arcuate top portion which is formed with a lateral through hole 310. In addition, the through hole 310 is configured to receive the insertion of a support shaft 424 of the array 400 therein. The main body 420 of the array 400 has a concave portion with recessed sides 426 for holding the support shaft 424 therebetween so that the link 300 is restricted from moving side to side. Line B is an axis along the center line of the support shaft 424.

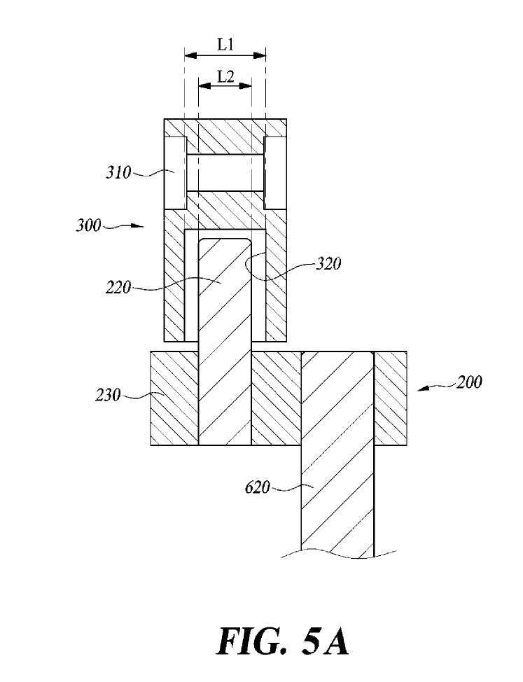

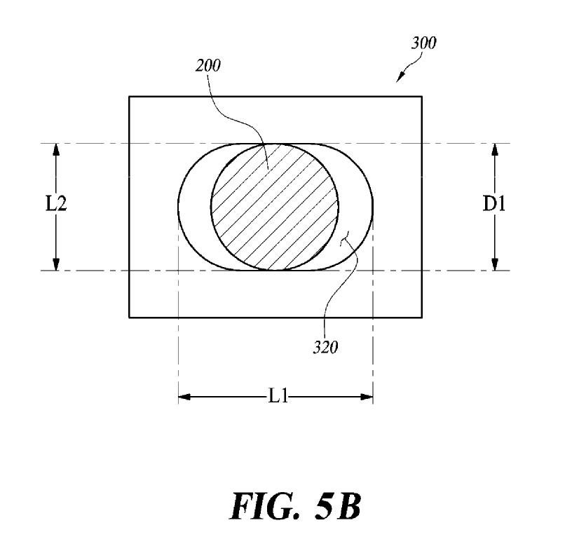

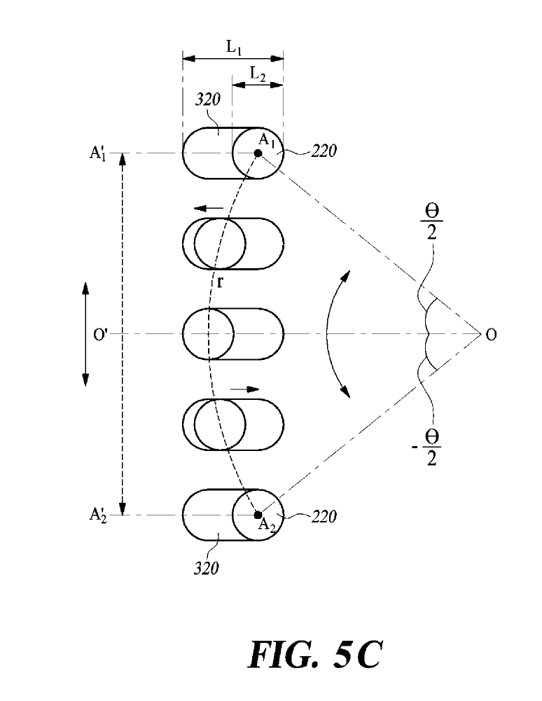

In particular, as shown in FIG. 3, the link 300 has a bottom formed with a guide groove 320 corresponding to the insertion pin 220 of the swivel arm 200. The guide groove 320 is configured to convert the rotation of the insert pin 220 into the thrust motion of the link 300, and it is designed as an oval with its long axis oriented laterally as shown in FIG. 5B. The insertion pin 220 is inserted into the guide groove 320 which defines the travel of the insertion pin 220 for actuating the link 300 in response to swivel motions of the swivel arm 200.

As shown in FIG. 5A, the oval guide groove 320 is configured to have a total length L1 of the long axis in the side to side direction, which is greater than diameter L2 of the insertion pin 220 so that the guide groove 320 may travel therein up to a distance L1-L2. At the same time, as shown in FIG. 5B, the short axis of the oval guide groove 320 has an appropriate total front-to-back length D1 which is substantially the same as or slightly greater than diameter L2 of the insertion pin 220 so that both front and rear ends of the insertion pin 220 are in contact with the guide groove 320.

FIG. 5C is a diagram showing in a plan view a thrust translation of the link 300 by the rotation of the insert pin 220, where the oval guide groove 320 is depicted with its long axis exaggerated for convenience of description.

In response to the rotation of the shaft 620 as described above, the insertion pin 220 swivels about a center point O from a line segment OA₁ to a line segment OA₂ along a trajectory ‘r’ in a range of rotational angle θ. Rotational angle θ also means the angle of rotation that the swivel arm 200 describes about the axis A.

At this time, the insertion pin 220 abuts against the guide groove 320 and is allowed to move as described above, a distance of up to L1-L2, which provides a slack for the insertion pin to move in the right-left directions in the guide groove 320 while swiveling along the trajectory ‘r’.

At this time, as shown in FIG. 2, with the through-hole 310 of the link 300 being substantially coextensive with the support shaft 424 of the array 400, and the main body of the array 400 blocking the link 300 with substantially no clearance allowed to move in the right-left directions in the drawing, the guide groove 320 may not follow suit the same swivel movements of the insert pin 220, but it performs linear movements or translates between a position A₁′ and a position A₂′ as viewed in plan view in the arrow direction.

In other words, with the insertion pin 220 being movable in the right-left directions in the guide groove 320, the insertion pin 220 in its swivel movement pushes the guide groove 320 constrained in its lateral movement, resulting in a thrust converted to a smooth linear movement of the link 300 without imposing strains between the components.

The guide groove 320 may have a height equalling that of the insert pin 220, but the guide groove 320 may be configured to be slightly higher than the insert pin 220 for a smooth motion transmission.

As a thrust mechanism, the insertion pin 220 in some embodiments has been described as being in constant abutment in the front-back direction with the guide groove 320, although the present disclosure is not limited to this arrangement and may be variously changed including forming a slight gap in the front-back direction between the insertion pin 220 and the guide groove 320 as long as they are arranged to convert the rotational force into the thrust motion.

With the insert pin 220 performing the swivel operation (thus, the interlocking movements of the link 300), the drive motor may be controllably operated in various ways for obtaining three-dimensional imaging by combining the movement of the insert pin 220 from position A₁ to position A₂, its movement about the reference point at O to either position A₁ or position A₂ from the midpoint between position A₁ and position A₂, and the reversed movements thereof.

The following describes the configuration and operation of the array 400 of some embodiments with reference to FIGS. 2, 3 and 7. The array 400 of some embodiments includes an arcuate front portion 410 and a main body 420 formed laterally with a hinge coupler 422, wherein the front portion 410 comprises a backing member having a plurality of piezoelectric elements which are arranged in parallel with excitation electrodes and installed with an acoustic matching layer and another layer of acoustic lens. While the array 400 rotates, the front portion 410 having the respective structural features as above emits ultrasonic waves in the forward direction.

A hinge coupler 422 is rotatably coupled to a receiving portion 122 formed on the upper portion of each side wall 120 at a position corresponding to the hinge coupler 422, as shown in FIG. 3. Thus, the array 400 is rotatable about the common axis (C in FIG. 2) of the hinge coupler 422 and the receiving portion 122. In this embodiment, it should be noted that array 400 itself is not directly movable in the front-back direction as the link 300 is.

The main body 420 of the array 400 has its support shaft 424 inserted in the through hole 310 of the link 300, and thus the link 300 is operatively linked with the array 400. Disposed in the concave portion of the main body 420, between the recessed side walls 426, the support shaft 424 serves as a stop for restraining the link 300 from moving side to side.

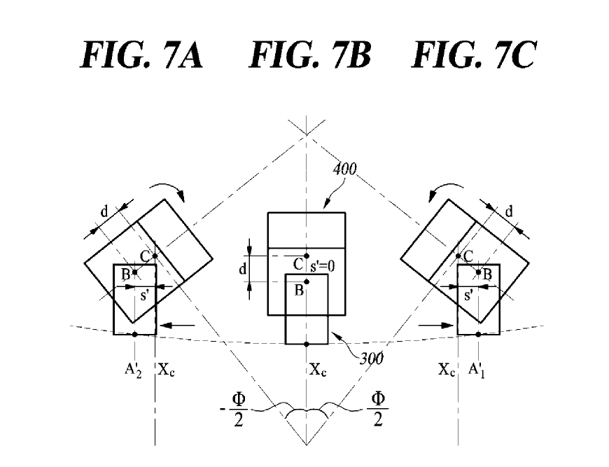

The following description addresses the swivel operation of the array 400 due to the rotation of the arm 200, with reference to FIG. 5C and FIG. 7.

In FIG. 7, the link 300 exhibits a reciprocating motion in a pendulum-like fashion, while the array 400 is not directly driven except for swiveling about the axis C.

For instance, the swivel arm 200 is in the initial position O′ of FIG. 5(c) with the link 300 and the array 400 assumed to be positioned in the first status at FIG. 7(b). When the motor operates to turn the swivel arm 200 counterclockwise along the trajectory ‘r’, the link 300 shifts toward position A₂′, which generates a thrust force that urges the array 400 to follow a route equivalent to the trajectory of the swivel arm 200. However, the array 400 is stopped from making the movement of its own by the hinge coupler 422 captured within the receiving portion 122, and with the resultant stopping force overcoming the thrust force, the array 400 can perform no linear movement but clockwise rotational movement about axis C in a direction opposite to the moving direction of the link 300, as shown in FIG. 7(a).

Conversely, when the motor operates to turn the swivel arm 200 at position O′ clockwise along the trajectory ‘r’, the link 300 shifts toward position A₁′, which generates a thrust force that urges the array 400 to follow a route equivalent to the trajectory of the swivel arm 200 on the same plane. However, the array 400 is immobilized by its own hinge coupler 422 captured within the receiving portion 122, and with the resultant stopping force overcoming the thrust force, the array 400 is still incapable of performing linear movement but counterclockwise rotational movement about axis C in a direction opposite to the moving direction of the link 300, as shown in FIG. 7(c).

At this time, the distance ‘d’ between axis B and axis C is fixed, but the rotational angle (φ) of the array 400 varies with the vertical distance (s′) between a reference axis Xc extending perpendicular to axis C and axis B. Reference axis Xc is in the same direction as axis A shown in FIG. 2. It follows from the foregoing that the distance (s′) is is the same as the linear distance for the link 300 to move by the thrust force received from the swivel arm 200. Before operation, the motor is in the initial neutral state at FIG. 7(b) having neither rotations of the array 400 and the swivel arm 200 nor a linear movement of the link 300.

Axis C is a fixed axis, axis B is a translation axis, and the axial distance ‘d’ between two axes B and C is the same, which causes the link 300 to move along an arc of the imaginary circle with a radial distance that preferably equals to the axial distance ‘d’. For example, when the link 300 shifts first from FIG. 7(b) to FIG. 7(a) and then back and forth between FIG. 7(a) and FIG. 7(c), the link 300 repeats to perform a linear motion as viewed in plan view and a rotary motion in a pendulum-like fashion as viewed in side view, as described above. This allows the array 400 under the thrust of the link 300 to swivel through a predetermined angle (φ) between a first position and a second position while irradiating and receiving ultrasound waves.

As is apparent to those skilled in the art, the radius of curvature of the pendulum-like rotary motion of the link 300 is appropriately set to absorb the vertical distance variations between the two axes B and C (with axes B-C distance constant).

The array 400 has its range of swivel motion appropriately set to be as wide as possible within its rotational angle (φ) defined by one end stop at FIG. 7(a) and the other end stop at FIG. 7(c) with the reference line of the array 400 as the center, within a range that allows to capture three-dimensional images, and within a range that allows to capture four-dimensional images upon adding time as a variable.

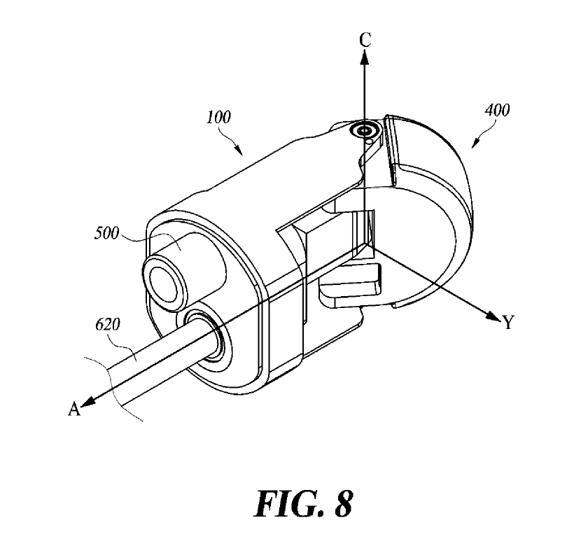

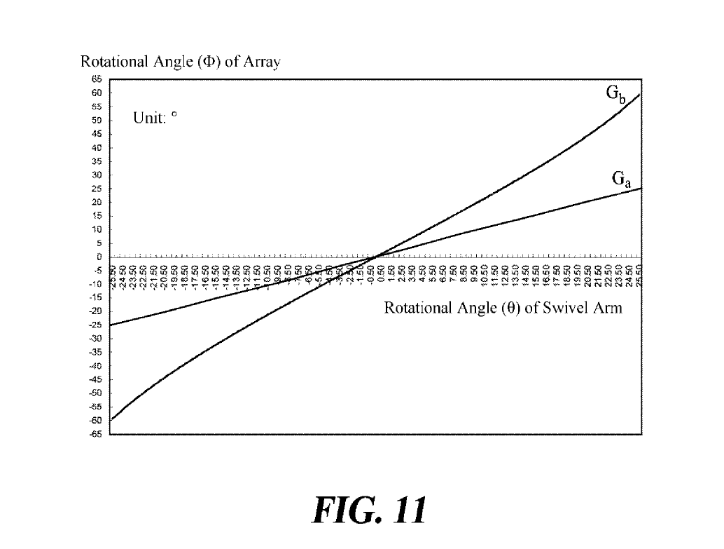

The operations of the swivel arm 200 and the array 400 according to some embodiments will be further described with reference to FIGS. 8 to 10. FIG. 11 is a graph for explaining analysis results of the rotation angles of the swivel arm 200 and the array 400 according to some embodiments.

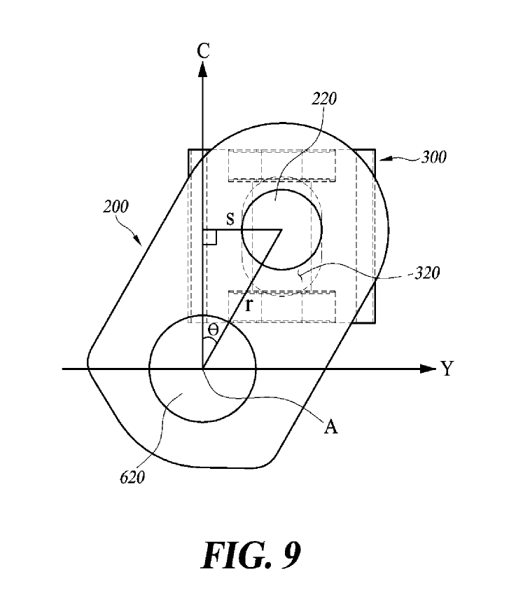

In FIG. 9, the link 300 moves in a direction parallel to axis Y where the moving distance (s) is changed, and thus the rotational angle (θ) is a variable, and the distance (r) between axis A and the center line of the insertion pin 220 is constant. Therefore, the following relationship in Equation 1 holds true.

S=r sin θ Equation 1

r: distance between axis A and the center line of the insertion pin 220

θ: rotational angle of the swivel arm 200.

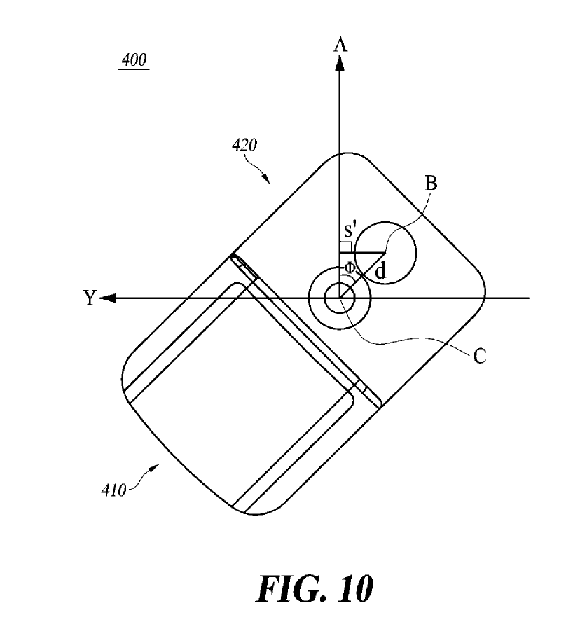

Now, in FIG. 10, the link 300 moves in the direction parallel to axis Y as mentioned in FIG. 7 with the moving distance (s′) changed, and thus the rotational angle (θ) of the array 400 is a variable, and the distance (d) between axis Band axis C is constant. Therefore, the following relationship in Equation 2 holds true.

S′=d sin Φ Equation 2

d: distance between axis B and axis C

φ: rotational angle of the array 400

Herein, the moving distance (s) represents the moving distance of the link 300 at its bottom face, and the the moving distance (s′) represents the same at its top surface, leading to s=s′ where s may be used as a parameter to express the relationship between the rotational angle (θ) of the swivel arm 200 and the rotational angle (φ) of the array 400 by the following Equation 3.

Φ=sin⁻¹(s/d)=sin⁻¹(r sin θ/d)=sin⁻¹((r/d)sin θ) Equation 3

In Equation 3, when three of the four parameters of r, d, θ and φ are determined, the remaining one parameter is automatically calculated. In practice, the rotational angle of the array 400 in contact with the target of the ultrasonic scanning is so significant value to be premised first, followed by determining the distances (r and d) according to the design requirements, which leaves the rotational angle (θ) of the swivel arm 200 as the control variable. Controlling the rotational angle (θ) is carried out by controlling the motor rotation amount and the direction of rotation of the driving unit 600. As a result, the motor-controlled manipulation of rotational speed, rotational angle and rotational direction of the swivel arm 200 enables the precise control of the rotation of the array 400. This allows an ultrasonic transducer to be implemented for the precise provision of four-dimensional images by adding temporally changing images to a three-dimensional spatial image.

Although distances (r and d) can also be appropriately selected by any design methods, an appropriate exemplary method is to set the distances based on the value of r/d by the following equation.

1≦r/d≦2 Equation 4

If r=d, φ=θ and a graph showing the changes of the φ value corresponding to the change in the θ value is a primary straight line, which is shown in a graph (Gₐ) of FIG. 11.

If r/d=2, example values of r, d, θ and φ are shown in the following Table 1.

TABLE 1

| | | |
|---|---|---|
| Range of | Rotational Angle (θ) of Swivel Arm 200 | ±25.66° |
| Rotational | Rotational Angle (Φ) of Array 400 | ±60.00° |
| Angles | | |
| Distance | Distance (r) between Axis (A) and the | 6 mm |
| | Center Line of Insertion Pin 220 | |
| | Distance (d) between Axis (B) and the | 3 mm |
| | Center Line of Axis (C) | |

At this time, φ=sin⁻¹(2 sin θ) in Equation 3, which is satisfied by a relationship as shown by a graph (G_{b}) of FIG. 11. The range of rotational angle (8) of the swivel arm 200 is ±25.66°, the range of rotational angle (φ) of the array 400 is ±60.00°. In other words, when the swivel arm 200 is rotated up to 25.66°, the array 400 is rotated by 60°.

Similarly, in the range 1≦r/d≦2, that is, between the graph (Gₐ) and graph (G_{b}), the r and d values may be varied to derive values spread linearly mutually between the rotational angles of the swivel arm 200 and those of the array 400, so as to apply the mutually linearly spread rotational angle values to the present disclosure.

In general, the straighter the graph showing the changes of the φ value corresponding to the change in the θ value, the better the operation performance of the ultrasonic transducer. The straighter the graph, the more linearly the array 400 under the thrust of the swivel arm 200 swivels without abrupt speed change in response to the rotational speed of the swivel arm 200, and therefore controlling the rotational angle of the swivel arm 200 provides a precisely controlled rotation of the array 400.

Driving the motor of the driving unit 600 for controlling the swivel arm 200 may be achieved in any way, such as by installing an on-off button on the housing 610, linking the driving unit 600 with a system (not shown) for controlling thereof, or providing a remote control.

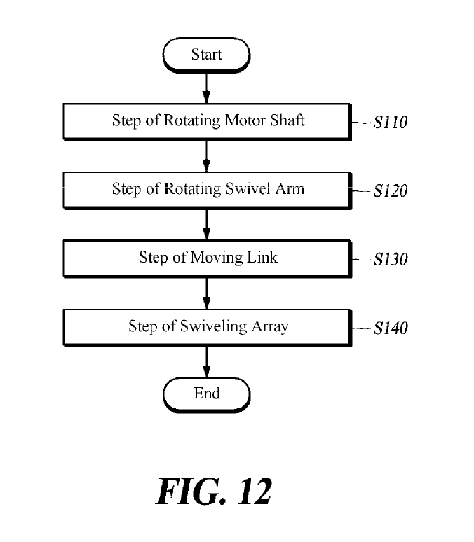

FIG. 12 is a flowchart showing the operating sequence of the ultrasonic transducer according to at least one embodiment of the present disclosure.

A method of operating the ultrasonic transducer includes steps of rotating the motor shaft 620 (S110), rotating the swivel arm 200 (S120), moving the link 300 (S130) and swiveling the array 400 (S140).

In Step S110 of rotating the motor shaft 620, it rotates about the axis (A) upon receiving an actuating force from the motor 610.

In Step S120 of rotating the swivel arm 200 that is linked with one side of the shaft 620, the swivel arm 200 rotates about the axis (A) in response to the rotation of the motor shaft 620.

In Step S130 of moving the link 300, it receives thrust from its linked swivel arm 200 to make planar linear movements and perform a pendulum motion at the same time about the axis (C).

In Step S140 of swiveling the array 400, it swivels about the axis (C) in response to the the linear movements of the link 300.

A limited number of embodiments are disclosed as above, although other variations thereof are also envisioned. The technical details of the aforementioned embodiments may be combined in many different forms unless they are incompatible with each other, whereby allowing new embodiments to be implemented.

Example embodiments of the present disclosure are based on the idea that the thrust from the rotation of a swivel member is utilized for causing a linking member to move and in turn cause the array to swivel, and those embodiments have many variations without departing from the scope of the idea. For example, without involving the base of the housing or such members, the driving power from the driving member can be delivered directly to the rotating member.

A possible modification provides an arrangement wherein the linking member performs the same rotational motion as with the swivel member, and the array follows a predetermined linear path and performs a swivel movement at the same time.

The components of the shapes, sizes, positions and the like as illustrated in some embodiments of the present disclosure are not intended to restrict or limit the scope of the present disclosure, and they are subject to any possible change, deletion, and substitution to an ordinary degree by those skilled in the art, who would understand the scope of the claimed disclosure is to be limited by the claims and equivalents thereof.

1. An ultrasonic transducer, comprising:
a swivel member configured to rotate by an actuating force and have a thrust mechanism;
a linking member configured to be connected to the swivel member and make a movement along a pendulum motion path by a thrust generated by the thrust mechanism of the swivel member; and
an array configured to be connected to the linking member and rotate in a range of a predetermined angle by a thrust generated by the movement of the linking member.

2. The ultrasonic transducer of claim 1, further comprising:
a driving unit configured to transmit the actuating force, the driving unit comprising a motor.

3. The ultrasonic transducer of claim 1, wherein the thrust mechanism of the swivel member comprises an insertion pin , and the linking member includes a guide groove configured to receive the insertion pin.

4. The ultrasonic transducer of claim 3, wherein the guide groove has a length which is greater than a diameter of the insertion pin.

5. The ultrasonic transducer of claim 4, wherein the array includes a stop for restraining the linking member from moving side to side.

6. The ultrasonic transducer of claim 4 or 5, wherein the linking member is configured to linearly move, as viewed in plan view, by the thrust generated by the thrust mechanism according to a swivel motion of the swivel member.

7. The ultrasonic transducer of claim 6, wherein the insertion pin of the swivel member is configured to, according to the swivel motion, translate within the guide groove of the linking member while thrusting the linking member , to thereby allow the linking member to linearly move.

8. The ultrasonic transducer of claim 1, wherein the linking member has a through hole, and the array comprises a support shaft inserted in the through hole.

9. The ultrasonic transducer of claim 1, further comprising a housing configured to receive the array,
wherein the array further comprises a coupler coupled to the housing at sides of the housing for allowing the array to swivel about the coupler.

10. The ultrasonic transducer of claim 9, wherein the coupler has an axis about which the array is rotated by the movement of the linking member.

11. The ultrasonic transducer of claim 1, wherein the predetermined angle of the array ranges from −60 to +60 degrees.

12. An ultrasonic transducer, comprising:
a swivel member configured to rotate by an actuating force and include an insertion pin;
a linking member configured to include a guide groove for receiving the insertion pin of the swivel member and have a through hole; and
an array configured to include a support shaft that is inserted in the through hole of the linking member.

13. The ultrasonic transducer of claim 12, wherein the insertion pin of the swivel member applies a thrust to the guide groove so that the linking member perform a linear motion as viewed in plan view and a pendulum motion as viewed in side view.

14. The ultrasonic transducer of claim 13, wherein the array is provided with a coupler having an axis about which the array is rotated in a predetermined angle range by a thrust generated by the linear motion of the linking member.

15. The ultrasonic transducer of claim 14, wherein the following relationship holds:
s=r sin θ
wherein:
s represents a linear moving distance of the linking member;
r is a distance between a rotational axis of the swivel member and a center line of the insertion pin;
θ is a rotational angle of the swivel member.

16. The ultrasonic transducer of claim 14, wherein the following relationship holds:
s′=d sin φ
wherein:
s′ represents a vertical distance between an axis extending perpendicular to the axis of the coupler of the array and the support shaft;
d is a distance between the axis of the coupler and a center line of the support shaft;
φ is a rotational angle of the array.

17. The ultrasonic transducer of claim 16, wherein the following relationship holds:
φ=sin⁻¹(s/d)=sin⁻¹(r sin θ/d)=sin⁻¹((r/d)sin θ)
wherein:
s=s′.

18. The ultrasonic transducer of claim 17, wherein 1≦r/d≦2.

19. The ultrasonic transducer of claim 17 or 18, wherein φ ranges from −60 to +60 degrees as measured with reference to the axis extending perpendicular to the axis of the coupler.

20. An ultrasonic transducer, comprising:
a swivel arm configured to rotate by an actuating force and include an insertion pin;
a link configured to include a guide groove for receiving the insertion pin of the swivel arm and have a through hole; and
an array configured to include a support shaft that is inserted in the through hole of the link, wherein
the guide groove has a length which is greater than a diameter of the insertion pin, and the array includes a stop for restraining the link from moving side to side,
the ultrasonic transducer further comprises a housing configured to receive the array,
the array further comprises a coupler coupled to the housing at sides of the housing,
the insertion pin of the swivel arm rotates for causing the link including the guide groove to perform a linear motion, and
upon receiving a thrust generated by the linear motion of the linking member, the array is rotated in a predetermined angle range about an axis of the coupler.

21. A method of operating an ultrasonic transducer, the method comprising:
rotating a swivel member with an actuating force from a driving unit;
moving a linking member linearly as viewed in plan view along a pendulum motion path with a thrust generated by the rotation of the swivel member, through a thrust mechanism provided in the swivel member; and
rotating an array connected to the linking member in a predetermined angle range with a thrust generated by the linear motion of the linking member.

22. The method of claim 21, wherein the linear motion of the linking member is performed by:
providing the swivel member with an insertion pin and providing the linking member with a guide groove which is larger than a diameter of the insertion pin, and
causing the insertion pin to thrust the guide groove.

23. The method of claim 22, wherein the rotating of the array in the predetermined angle range comprises:
rotating the array about a predetermined axis with a thrust generated by the linear motion of the linking member.
